(19) United States
(12) Patent Application Publication
(10) Pub. No.: US 2023/0285053 A1
(43) Pub. Date: Sep. 14, 2023

(54) Bone Fixation System for Spinal Stabilization

(71) Applicant: Kristen M. McLaughlin, Glen Mills, PA (US)

(72) Inventor: John J. Sinisi, Warminster, PA (US)

(21) Appl. No.: 18/319,417

(22) Filed: May 17, 2023

Related U.S. Application Data
(63) Continuation-in-part of application No. 17/523,530, filed on Nov. 10, 2021.

(51) Int. Cl. A61B 17/70

(52) U.S. Cl. CPC A61B 17/7032; A61B 17/7035; A61B 17/86

(57) ABSTRACT

A bone fixation system where an alignment rod is engaged by pedicle screws. The alignment rod has sections of buttress thread. Each pedicle screw has a threaded shaft that supports a slotted receptacle. A set screw threads into the slotted receptacle. A spring plates is interposed between the set screw and the alignment rod. The spring plates contain protrusions that engage the sections of buttress thread on the alignment rod. The threaded shaft of the pedicle screw has an upper section and a lower section. A threaded connecting rod interconnects the sections. The upper section, lower section and threaded connecting rod all have external threads of the same thread pitch.

### RELATED APPLICATIONS

This application is a continuation-in-part of U.S. patent application Ser. No. 17/523,530, filed Nov. 10, 2021.

### BACKGROUND OF THE INVENTION

### 1. Field of the Invention

In general, the present invention relates to the bone fixation systems that are used to adjust and/or stabilize the vertebrae of the spine. More particularly, the present invention relates to the structure of pedicle screws and alignment rods used in bone fixation systems.

### 2. Prior Art Description

It is not uncommon for a person to be born with some form of a skeletal irregularity or to develop some skeletal irregularity as they age. Most irregularities are minor and do not adversely affect a person's wellbeing. However, some skeletal irregularities, especially spinal irregularities, can cause pain and can limit range of motion. These irregularities can result from trauma, age, disc degeneration, and disease. Often, the treatment for such irregularities is the repositioning and/or immobilizing of a portion of the spine. This treatment commonly involves affixing a plurality of pedicle screws to the vertebrae and interconnecting the pedicle screws with one or more elongated rods. The problem that there are differences in the size and location of vertebrae and the degree to which vertebrae need to be adjusted in each person.

Consider the spinal irregularities caused by scoliosis. Scoliosis may cause deviations in all three directions or planes of the spine, i.e. frontal (coronal), lateral (sagittal) and transversal (axial). The list of clinical problems associated with scoliosis is extensive. It includes alteration of normal gait associated with pelvic obliquity, distortion of abdominal and chest organs, and the associated alteration of functional capabilities of the organs.

For idiopathic types of scoliosis, there is no congenital anomalies of the vertebrae. Therefore, evaluation of treatment is complicated and often empirical. Historically, the treatment is the surgical correction of the spine. The principles of surgical correction include two basic steps. The first step comprises the acute correction of spinal deformity during the surgery and the insertion of a holding device. The second step comprises the solid fusion of vertebral bodies in the position of gained correction, by insertion of bone graft during the same surgical procedure. Idiopathic scoliosis is not an acute illness, and with time, vertebrae become secondarily deformed. Surgeons who treat scoliosis know about-deformed shape of scoliotic vertebrae. For correction of such scoliotic deformity, acute manual correction of deformity during surgery is used. Positioning is maintained using pedicle screws that attach holding rods or plates to the vertebral body or vertebral prominences. When a surgeon manipulates the pedicle screws, rods and plates, the degree of fine adjustment is limited. Accordingly, there are pedicle screws, rods and plates that have been designed to be finely adjusted after they are surgically implanted. In this manner, a surgeon can make fine adjustments after all the hardware is anchored in place and the effects of the hardware can be observed.

Traditionally, pedicle screws are used to mechanically engage the vertebrae. The pedicle screws have anchor heads that are shaped to engage a rod or plate. In the prior art, there are pedicle screws that can be adjusted in length within the body. Such prior art pedicle screws are exemplified by U.S. Pat. No. 9,089,371 to Faulhaber. These prior art pedicle screws have different sections that are internally threaded together. As a result, if one section is rotated relative to a second section, the pedicle screw will either elongate or retract. A problem associated with such prior art pedicle screws is that some of the internal threading used for length adjustment is exposed on the exterior of the pedicle screw as it is elongated. The pitch of the exposed internal threading does not match the pitch of the threading on the exterior of the pedicle screw. As a result, when the pedicle screw is rotated into bone, or removed from bone, the bore in the bone is exposed to threading having two different pitches. This causes the bone bore to become double threaded as the pedicle screw turns. This makes the pedicle screw more difficult to install and/or remove. Furthermore, the double threading causes the lesser of the threads to strip through the bone as the more dominant threading turns. This widens the bone bore and degrades the mechanical connection. Another problem associated with adjustable pedicle screws in the prior art is that the pedicle screws are difficult to adjust so that the screws engage the cortical bone on opposite sides of the vertebral body. Rather, many prior art pedicle screws anchor into the porous cancellous bone within the vertebral body, which creates a far weaker bond than would connection with the stringer cortical bone.

In addition to adjusting the length of the pedicle screws, the engagement between the rods and the pedicle screws must also be adjustable. In the prior art, there are many rods that are machined or textured in some manner to mechanically interconnect with the head of a pedicle screw in a manner that is adjustable. Such prior art systems are exemplified by U.S. Pat. No. 8,088,149 to White. A problem associated with such adjustment systems is that the head of the pedicle screw only engages the adjustment rod when tightened. When correcting a curvature in the spine, a surgeon must reposition the vertebrae. This requires a surgeon to maintain forces on the vertebrae as the alignment rod is anchored in place. As such, a surgeon must position and maintain tension on the adjustment rod while tightening the adjustment rod to the pedicle screws. This is a complicated task. Accordingly, a need exists in the art for an adjustable pedicle screw that better engages the cortical bone on a vertebral body. A need also exists for a pedicle screw/adjustment rod system that makes it easier to maintain the adjustment rod in tension prior to being firmly anchored into a fixed position. These needs are met by the present invention as described and claimed below.

### SUMMARY OF THE INVENTION

The present invention is a bone fixation system where an alignment rod is engaged by pedicle screws. The alignment rod has a top surface upon which sections of buttress thread are formed.

Each pedicle screw has a threaded shaft that supports a slotted receptacle. The slotted receptacle defines a slot that is sized to receive an alignment rod. The slot has opposing walls that are internally threaded. A set screw that threads into the opposing walls, wherein the set screw can be selectively advanced into the slot to an adjustment position and a deeper locked position.

A spring plate is interposed within the slot between the set screw and the alignment rod. The spring plate contains protrusions that engage the sections of buttress thread on the alignment rod when the protrusions are biased against the alignment rod by the set screw. The spring plate prevents the alignment rod from moving in more than one direction through the slot when the set screw is in its adjustment position. Furthermore, the spring plate prevents the alignment rod from moving through the slot in any direction when the set screw is in its locked position.

The threaded shaft of the pedicle screw has an upper section and a lower section. A threaded connecting rod interconnects the upper section and the lower section. The threaded connecting rod is exposed to different degrees when the threaded shaft is adjusted in length and the lower section is turned relative to the upper section. The upper section, lower section and threaded connecting rod all have external threads of the same thread pitch.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is a perspective view of a segment of a bone fixation system that utilizes adjustment rods and pedicle screws;

FIG. 2 is a side view of the adjustment rod shown in FIG. 1;

FIG. 3 is a cross-sectional view of the alignment rod shown in FIG. 2 viewed along section line 3-3;

FIG. 4 is an exploded view of the exemplary embodiment of FIG. 2;

FIG. 5 is a cross-sectional view of the exemplary embodiment of FIG. 1;

FIG. 6 is a cross-sectional view of a vertebral body being engaged by the pedicle screw of the present invention bone fixation system.

FIG. 7 is a cross-sectional view of an anchor head assembly of the pedicle screw in a locked position engaging an alignment rod; and

FIG. 8 is a cross-sectional view of an anchor head assembly of the pedicle screw in an adjustment position partially engaging an alignment rod.

### DETAILED DESCRIPTION OF THE DRAWINGS

Although the present invention system can be embodied in many ways, only one exemplary embodiment is illustrated. The exemplary embodiment is being shown for the purposes of explanation and description. The exemplary embodiment is selected in order to set forth one of the best modes contemplated for the invention. The illustrated embodiment, however, is merely exemplary and should not be considered a limitation when interpreting the scope of the claims.

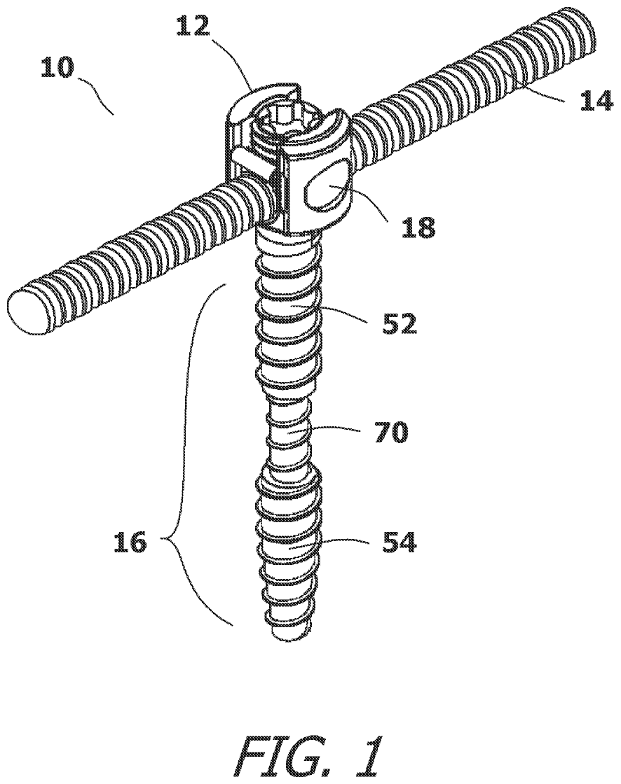

Referring to FIG. 1, a bone fixation system 10 is shown. The bone fixation system 10 consists primarily of a specialized pedicle screw 12 and a specialized alignment rod 14. The pedicle screw 12 has a shaft assembly 16 that terminates at one end with an anchor head assembly 18. The anchor head assembly 18 is designed to selectively receive and engage the alignment rod 14. As will be explained, the anchor head assembly 18 works in concert with the alignment rod 14 so that the alignment rod 14 can be pulled in one direction while the anchor head assembly 18 automatically prevents the tensioned alignment rod 14 from moving back in the opposite direction.

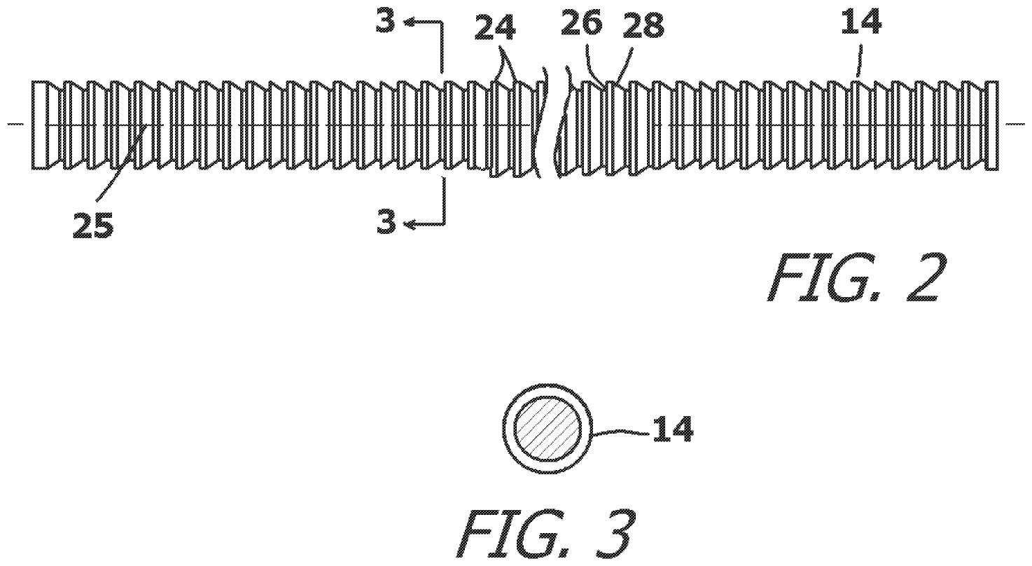

Referring to FIG. 2 and FIG. 3 in conjunction with FIG. 1, it can be seen that the alignment rod 14 can be any length as needed by a surgeon. The alignment rod 14 can be either straight or curved. The alignment rod 14 preferably has a rounded profile. However, polygonal shapes can also be used. In a preferred embodiment, the alignment rod 14 has a round profile and is machined with buttress threading 24. The alignment rod 14 has a midline 25 that follows the curvature of the alignment rod 14. Each segment of the buttress threading 24 has a vertical surface 26 and a sloped surface 28. The vertical surface 26 is perpendicular to the midline 25 of the alignment rod 14. The sloped surfaces 28 slope at a descending angle away from the vertical surface 26 in a first direction. All the sloped surfaces 28 slope in the same first direction at the same pitch angle. In the shown embodiment, the buttress threading 24 extends around the alignment rod 14. However, in alternate embodiments, the buttress threading can be limited to arc angles that only partially extend around the alignment rod 14.

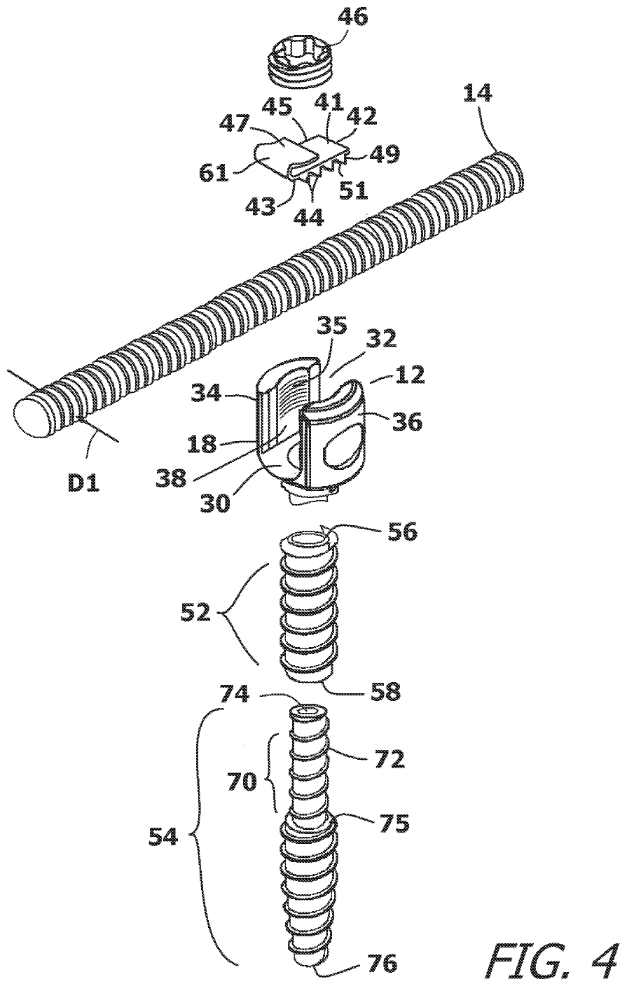

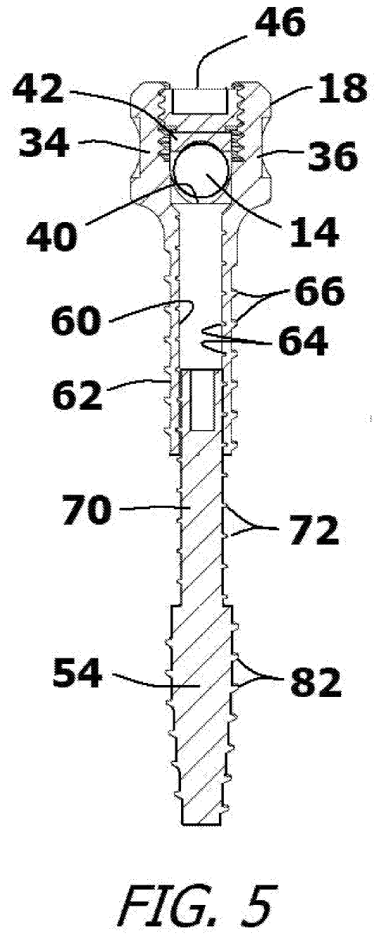

Referring to FIG. 4, FIG. 5, in conjunction with FIG. 1, it can be seen that the anchor head assembly 18 of the pedicle screw 12 includes a slotted receptacle 30. The slotted receptacle 30 has an open top 32 and two opposing walls 34, 36 that are spaced apart by a slot 38. The slot 38 is accessible through the open top 32. The slot 38 is slightly wider than the diameter D1 of the alignment rod 14. The opposing walls 34, 36 are internally threaded. That is, the opposing walls 34, 36 have threads 35 on interior surfaces that face the slot 38. The slotted receptacle has a bottom surface 40 that is contoured to match the shape of the bottom surface 22 of the alignment rod 14. In this manner, the alignment rod 14 can be cradled in the slotted receptacle 30.

A spring plate 42 is provided as part of the anchor head assembly 18. The spring plate 42 has a width that is generally equal to the diameter D1 of the alignment rod 14. Accordingly, the spring plate 42 is sized to fit into the slot 38 between the opposing walls 34 36 atop the alignment rod 14. The spring plate 42 also has a length that is slightly longer than the slot 38. The spring plate 42 has a base section 45 and a cantilevered section 47 that is suspended over part of the curved spring section 47 by a 180 degree bend 61. The base section 45 has a top surface 41 and an underlying bottom surface 43. Buttress protrusions 44 are disposed on the bottom surface 43. The buttress protrusions 44 are sized and spaced to engage the buttress threading 24 on the top surface 20 of the alignment rod 14. The buttress protrusions 44 have vertical surfaces 49 that extend at a perpendicular from the base section 45 and slopes surfaces 51 that extend from the vertical surfaces 49. The curved bend 61 creates a spring force that is used to bias the buttress protrusions 44 against the buttress threading 24 of the alignment rod 14.

The cantilevered section 47 of the spring plate 42 is supported by the curved bend 61. The cantilevered section 47 is contacted by a set screw 46. The set screw 46 screws into the threading 35 on the opposing walls 34, 36 of the slot 38. As the set screw 46 is tightened, the set screw 46 presses against the cantilevered section 47 and begins to compress the curved bend 61. The curved bend 61 acts as a spring and redirects this contact force to the spring plate 42. This biases the spring plate 42 against the alignment rod 14. As a result, the buttress protrusions 44 on the spring plate 42 are biased into the buttress threading 24 of the alignment rod 14.

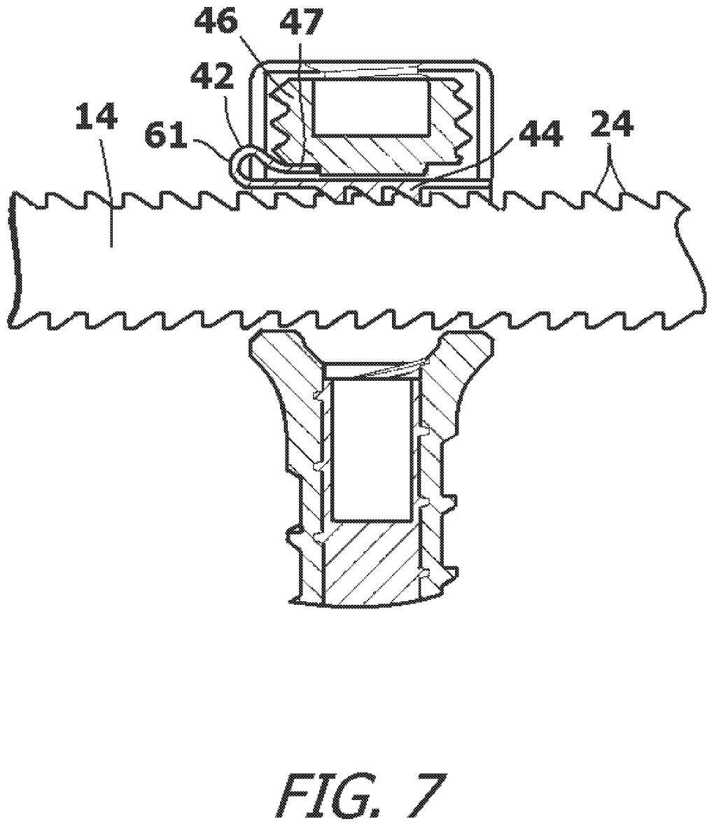

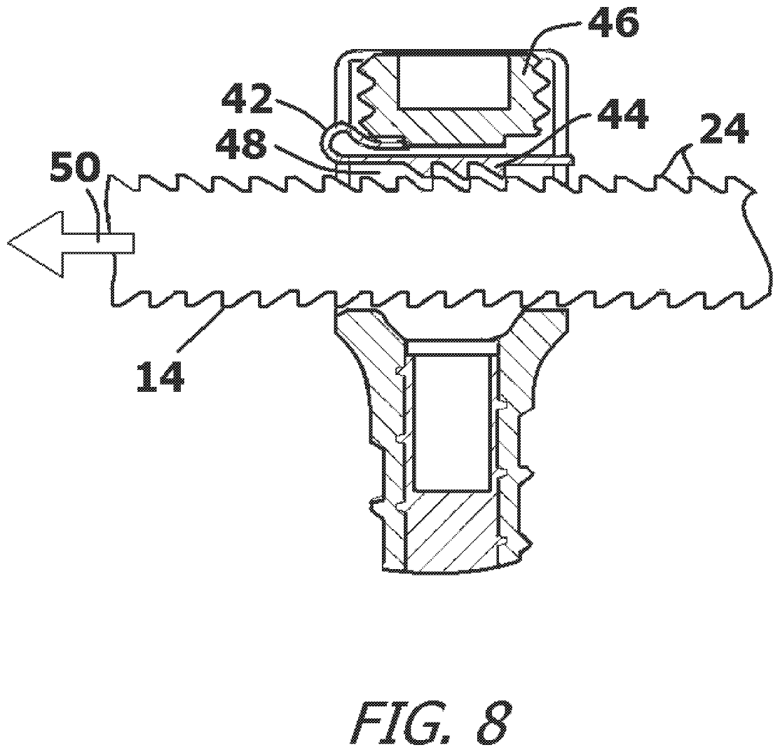

Referring to FIG. 7 and FIG. 8 in conjunction with FIG. 4 it can be seen that as the spring plate 42 is biased against the alignment rod 14 by the set screw 46, the curved bend 61 compresses and the buttress protrusions 44 on the spring plate 42 engage the buttress threads 24 on the alignment rod 14 to some degree. If the set screw 46 is firmly tightened, then the buttress protrusions 44 on the spring plate 42 fully engage the buttress threads 24 on the alignment rod 14. In this locked condition, the alignment rod 14 is firmly locked in place and cannot move independently in relation to the pedicle screw 12. See FIG. 5. However, as can be seen from FIG. 8, the set screw 46 can be set into an adjustment condition where the set screw 46 is not firmly tightened and a gap space 48 is created between the spring plate 42 and the alignment rod 14. If there is a gap space 48, the spring plate 42 has the ability to move in the gap space 48. If the gap space 48 is correctly sized, the buttress protrusions 44 on the spring plate 42 will engage the buttress threads 24 on the alignment rod 14 only if the alignment rod 14 is moved in the direction of arrow 50. Arrow 50 is also the same direction in which the sloped surfaces 28 of buttress threading 24 extends away from the vertical surfaces 26 of the buttress threading 24. If the alignment rod 14 is moved in the opposite direction of arrow 50, then the spring plate 42 floats atop the alignment rod 14 without engaging the alignment rod 14. It will therefore be understood that if the gap space 48 is properly sized using the set screw 46, then the alignment rod 14 can be pulled in one direction using one hand. This can place the alignment rod 14 in tension. Once properly positioned, the alignment rod 14 can be released and it will not move backward and release the tension. A surgeon can then lock the alignment rod 14 in place by tightening the set screw 46 to eliminate the gap space 48.

Referring back to FIG. 4 and FIG. 5, it can be seen that the pedicle screw 12 has a shaft assembly 16. The shaft assembly 16 has a first upper section 52, a second lower section 54 that are all vertically aligned. The lower section 54 includes a threaded connection rod 70. With the exception of an Allen key depression in the first end 74 of lower section 54, the lower section 54 of the pedicle screw 12 is fully solid. The solid structure of the lower section 54, provides the lower section 54 with great strength and durability.

The upper section 52 of the pedicle screw has a first open end 56 and an opposite second open end 58. The upper section 52 is hollow between the first open end 56 and the second open end 58. As such, the upper section 52 is tubular with both an interior surface 60 and an exterior surface 62. Both the interior surface 60 and the exterior surface 62 are threaded. As such, there are interior threads 64 and exterior threads 66. The exterior threads 66 have a certain thread pitch. The interior threads 64 have the same thread pitch, albeit with a smaller major diameter.

The first open end 56 of the upper section 52 is affixed to the slotted receptacle 30. The first open end 56 is accessible through the bottom 40 of the slotted receptacle 30. In this manner, an Allen key or similar tool can be inserted into the interior of the upper section 52 though the bottom of the slotted receptacle 30.

The second lower section 54 of the shaft assembly 16 is solid. The lower section 54 has a first end 74 and an opposite second end 76. The threaded connection rod 70 extends from the first end 74 of the lower section 54. The threaded connection rod 70 has a smaller diameter than does the lower section 54. As such, there is a stepped transition 75 at the point of attachment. The threaded connection rod 70 has external threads 72. The external threads 72 match the pitch, thread angle and diameter of the interior threads 64 on the interior surface 60 of the upper section 52. In this manner, the upper section 52 of the shaft assembly 16 threads onto the threaded connection rod 70. A keyed depression 80 is formed in the threaded connection rod 70. This keyed depression 80 can be accessed through the slotted receptacle 30. It will therefore be understood that a surgeon can advance a tool through the slotted receptacle 30 and the upper section 52. The tool can be used to turn the threaded connection rod 70 and the lower section 54 relative the upper section 52. As the lower section 54 turns relative to the upper section 52, the external threads 72 on the threaded connection rod 70 engage the interior threads 64 within the upper section 52. Accordingly, the turning of the threaded connection rod 70 and the lower section 54 will cause the lower section 54 and the upper section 52 to either move together or spread apart, depending upon the direction of rotation. As the lower section 54 and the upper section 52 move apart, the threaded connection rod 70 becomes more exposed and the overall length of the shaft assembly 16 increases. Conversely, as the lower section 54 and the upper section 52 thread together, the exposed length of the threaded connection rod 70 decreases as does the overall length of the shaft assembly 16.

The lower section 54 of the shaft assembly 16 has exterior threads 82. The exterior threads 82 match the pitch, thread angle and diameter of the exterior threads 66 on the upper section 52. Furthermore, when the lower section 54 is fully seated into the upper section 52, the exterior threads 82 of the lower section 54 seamlessly align with the exterior threads 66 on the upper section 52, as though the two sections 52, 54 were threaded as a single piece.

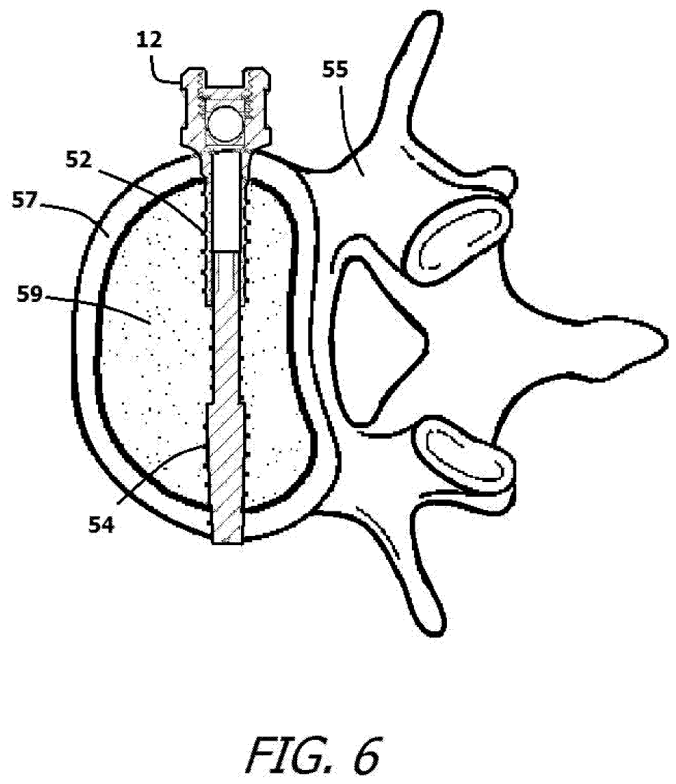

Referring to FIG. 6 in conjunction with FIG. 4 and FIG. 5, it will be understood that to utilize the bone fixation system 10, a surgeon sets the pedicle screw 12 into a vertebral body 55. The length of the pedicle screw 12 can be selectively adjusted by turning the lower section 54 of the shaft assembly 16 relative to the upper section 52. Fine adjustments can be made after initial implantation. The pedicle screw 12 is adjusted so that both the threaded upper section 52 and the threaded lower section 54 engage the cortical bone 57 on opposite sides of the vertebral body 55. The pedicle screw 12 passes through the cancellous bone 59 in the center of the vertebral body 55, however, most of the strength of the mechanical connection is provided by the denser cortical bone 57.

As the length of the pedicel screw 12 is increased, the threaded connection rod 70 becomes increasingly exposed within the vertebral body 55. However, the threaded connection rod 70 has the same thread pitch as is used on the exteriors of both the lower section 54 and the upper section 52. Accordingly, all exposed threading follows a single thread path through the vertebral body 55. Over time, bone grows against the pedicle screw 12. By utilizing a single pitch thread across all exposed threading, the pedicle screw 12 can be removed from the bone with far less damage to the bone since all threading follows a single thread path.

Once a pedicle screw 12 is implanted, the slotted receptacle 30 is oriented and adjusted to the proper elevation, via length adjustments to the pedicle screw 12. The surgeon selects the proper alignment rod 14 and sets the alignment rod 14 into the slotted receptacle 30 of the pedicle screw 12. The spring plate 42 and set screw 46 are installed above the alignment rod 14. The set screw 46 is advanced to an adjustment position where the spring plate 42 only engages the alignment rod 14 in one direction. The surgeon can therefore pull and tension the alignment rod 14 with one hand. Once the alignment rod 14 is properly tensioned, the alignment rod 14 can be released by the surgeon. The surgeon can then fully advance the set screw 46 form the adjustment position to a locked position, wherein the alignment rod 14 is locked in place.

It will be understood that the embodiment of the present invention that is illustrated and described is merely exemplary and that a person skilled in the art can make many variations to that embodiment. All such embodiments are intended to be included within the scope of the present invention as defined by the claims.

What is claimed is:

1. A bone fixation system, comprising:
an alignment rod having a top surface, wherein at least part of said alignment rod has buttress threading disposed along said top surface;
a pedicle screw having a threaded shaft that supports a slotted receptacle, wherein said slotted receptacle defines a slot sized to receive said alignment rod therethrough, said slot having opposing walls, wherein said opposing walls are internally threaded;
a set screw that threads into said opposing walls, wherein said set screw can be selectively advanced into said slot to an adjustment position and to a locked position;
a spring plate disposed between said set screw and said top surface of said alignment rod, said spring plate having a base section and a cantilevered section that are joined by a curved bend, therein suspending said cantilevered segment over at least part of said base section, wherein a plurality of parallel buttress protrusions extend from said base section, and wherein said cantilevered section is contacted by said set screw as said set screw is advanced toward said locked position therein causing said plurality of parallel buttress protrusions on said base section to engage said buttress threading on said alignment rod.

2. The system according to claim 1, wherein engagement between said plurality of parallel buttress protrusions on said base section and said buttress threading on said alignment rod limits said alignment rod to linear movement in only one direction when said set screw is advanced to said adjustment position.

3. The system according to claim 1, wherein engagement between said plurality of parallel buttress protrusions on said base section and said buttress threading on said alignment rod prevents any linear movement of said alignment rod when said set screw is advanced to said locked position.

4. The system according to claim 1, wherein said threaded shaft of said pedicle screw has a length that is selectively adjustable.

5. The system according to claim 4, wherein said threaded shaft has a hollow upper section and a solid lower section that thread together, wherein said length is adjusted by turning said upper section and said low section relative to each other.

6. The system according to claim 5, wherein said lower section includes a threaded connecting rod that engages said upper section, and wherein said threaded connecting rod is exposed in different amounts when said lower section is turned relative to said upper section.

7. The system according to claim 6, wherein said upper section, said lower section and said threaded connecting rod all have external threads of the same thread pitch.

8. The system according to claim 6, wherein said threaded connecting rod is rigidly affixed to said lower section of said threaded shaft.

9. The system according to claim 8, wherein said slotted receptacle is rigidly affixed to said upper section of said threaded shaft.

10. The system according to claim 9, wherein said upper section of said threaded shaft is hollow and said threaded connecting rod is accessible through said upper section.

11. The system according to claim 1, wherein said alignment rod has a smooth bottom surface that rests in said slotted receptacle.

12. The system according to claim 1, wherein said alignment rod has a midline and said buttress threadings have vertical surfaces that are perpendicular to said midline and a sloped surfaces that slope way from said vertical surfaces in a common first direction.
